(19) United States
(12) Patent Application Publication
(10) Pub. No.: US 2011/0190312 A1
(43) Pub. Date: Aug. 4, 2011

(54) Combinations Comprising Epothilones and Protein Tyrosine Kinase Inhibitors and Pharmaceutical Uses Thereof

(76) Inventors: Jerry Min-Jian Huang, Florham Park, NJ (US); Anandhi Ranganathan Johri, Short Hills, NJ (US); Ronald Richard Linnartz, Andover, NJ (US); Paul M.J. McSheehy, Loerrach-Stetten (DE)

(21) Appl. No.: 13/085,519

(22) Filed: Apr. 13, 2011

Related U.S. Application Data
(63) Continuation of application No. 11/718,927, filed on May 9, 2007.
(60) Provisional application No. 60/631,837, filed on Nov. 30, 2004.

(51) Int. Cl. A61K 31/496; A61K 31/427; A61K 31/4353; A61P 35/00

(52) U.S. Cl. 514/252.16; 514/365; 514/287

(57) ABSTRACT

The invention relates to a combination which comprises:
(a) an epothilone; and
(b) a protein tyrosine kinase inhibitor; and optionally
(c) a derivative of rapamycin; for simultaneous, separate or sequential use, in particular, for the delay of progression or treatment of a proliferative disease, especially cancer.

The invention relates to a combination which comprises:
(a) an epothilone; and
(b) a protein tyrosine kinase inhibitor; and optionally
(c) a derivative of rapamycin; for simultaneous, separate or sequential use, in particular, for the delay of progression or treatment of a proliferative disease, especially cancer.

The invention also relates to a pharmaceutical composition comprising such a combination and optionally at least one pharmaceutically acceptable carrier. The invention also relates to the use of such a combination for the preparation of a medicament for the delay of progression or treatment of a proliferative disease. The invention also relates to a commercial package or product comprising such a combination as a combined preparation for simultaneous, separate or sequential use; and to a method of treatment of a warm-blooded animal, especially a human.

### BACKGROUND OF THE INVENTION

The epothilones represent a class of microtubule stabilizing cytotoxic agents. See Gerth et al., J Antibiot, Vol. 49, pp. 560-563 (1966); or Hoefle et al., DE 41 38 042. They are 16-member macrolides containing seven, chiral centers and may also be characterized by various functionalities. For example, they may include other ring systems, such as an epoxide and/or a thiazole ring. They may have two free, derivatizable hydroxyl groups and the macrolide itself may comprise an ester linkage.

Cytotoxic agents are well-known for the treatment of tumors. The anti-tumor activity of many of these compounds relies on the inhibition of cell proliferation and consequent induction of apoptosis and cell death. The majority of cytotoxic agents exert their effects through interference of DNA and/or RNA syntheses. However, for certain cytotoxic agents, e.g., members of the taxane family, e.g., paclitaxel; and the epothilones, their activity is reliant on their interference with microtubule dynamics. Microtubules are an important and attractive target for development of novel anti-cancer formulations.

Protein tyrosine kinase inhibitors are widely used to inhibit protein tyrosine kinase activity in a variety of both benign and malignant diseases. Protein tyrosine kinase receptors play a key role in signal transmission in a large number of mammalian cells, including human cells, especially epithelial cells, cells of the immune system and cells of the central and peripheral nervous system. Most importantly, overexpression of these receptors has been observed in substantial fractions of many human tumors.

### SUMMARY OF THE INVENTION

It has now been found that surprisingly the administration of an epothilone with a protein tyrosine kinase inhibitor and optionally the addition of a derivative of rapamycin is useful for the treatment of a proliferative disease, especially cancer.

Accordingly, the present invention provides a combination, such as a combined preparation or a pharmaceutical composition, which comprises:
(a) an epothilone; and
(b) a protein tyrosine kinase inhibitor; and optionally
(c) a derivative of rapamycin,
   in which the active ingredients (a) and (b), and optionally (c), are present in each case in free form or in the form of a pharmaceutically acceptable salt, for simultaneous, concurrent, separate or sequential use in the treatment of a proliferative disease.

The term “a combined preparation”, as used herein, defines especially a “kit of parts” in the sense that the combination partners (a) and (b), and optionally (c), as defined above, can be dosed independently or by use of different fixed combinations with distinguished amounts of the combination partners (a) and (b), and optionally (c), i.e., simultaneously or at different time points. The parts of the kit of parts can then, e.g., be administered simultaneously or chronologically staggered, that is at different time points and with equal or different time intervals for any part of the kit of parts. Very preferably, the time intervals are chosen such that the effect on the treated disease in the combined use of the parts is larger than the effect which would be obtained by use of only anyone of the combination partners (a) and (b), and optionally (c). The ratio of the total amounts of the combination partner (a) to the combination partner (b), and optionally the addition of combination partner (c), to be administered in the combined preparation can be varied, e.g., in order to cope with the needs of a patient subpopulation to be treated or the needs of the single patient which different needs can be due to age, sex, body weight, etc. of the patients. Preferably, there is at least one beneficial effect, e.g., a mutual enhancing of the effect of the combination partners (a) and (b), and optionally (c), in particular, a synergism, e.g., a more than additive effect, additional advantageous effects, less side effects, a combined therapeutical effect in a non-effective dosage of one or both of the combination partners (a) and (b), and optionally (c), and very preferably, a strong synergism of the combination partners (a) and (b), and optionally (c).

Further, the invention provides the use of an epothilone, for use in combination with a protein tyrosine kinase inhibitor and optionally a derivative of rapamycin, for treatment of a proliferative disease, especially a malignant disease, such as cancer.

In the alternative, the invention provides the use of a protein tyrosine kinase inhibitor, for use in combination with an epothilone and optionally a derivative of rapamycin for treatment of a proliferative disease, especially a malignant disease, such as cancer.

In yet further aspects the invention provides a package comprising:

1. A package comprising an epothilone, together with instructions, for use in combination with a protein tyrosine kinase inhibitor and, optionally, a derivative of rapamycin for treatment of a proliferative disease, especially malignant disease, such as cancer; or
2. A package comprising either a protein tyrosine kinase inhibitor together with instructions for use in combination with an epothilone and, optionally, a derivative of rapamycin for treatment of a proliferative disease, especially malignant disease, such as cancer.

Diseases and conditions which may be treated in accordance with the present invention include breast cancer; ovarian cancer; cancer of the colon and generally the gastrointestinal tract including gastric cancer; cervix cancer; lung cancer, e.g., small-cell lung cancer and non-small-cell lung cancer; pancreas cancer; renal cancer; glioma; melanoma; head and neck cancer; bladder cancer; hepatocellular cancer; prostate cancer; and Kaposi's sarcoma.

Thus, in the present description, the terms “treatment” or “treat” refer to both prophylactic or preventative treatment, as well as curative or disease modifying treatment, including treatment of patients at risk of contracting the disease or suspected to have contracted the disease, as well as patient who are ill or have been diagnosed as suffering from a disease or medical condition.

### DETAILED DESCRIPTION OF THE PRESENT INVENTION

The epothilones of the present invention are derivatives of formula (I′)

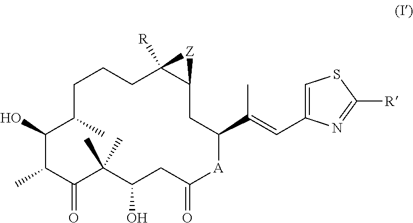

wherein A represents O or NR_{N}, wherein R_{N} is hydrogen or lower alkyl, R is hydrogen or lower alkyl, R′ is methyl, methoxy, ethoxy, amino, methylamino, dimethylamino, aminomethyl or methylthio, and Z is O or a bond, in free form or in the form of a pharmaceutically acceptable salt.

A more preferred embodiment are compounds of formula I

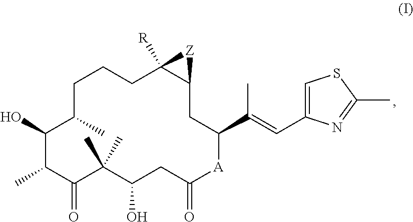

wherein
A represents O or NR_{N}, wherein R_{N} is hydrogen or lower alkyl;
R is hydrogen or lower alkyl; and
Z is O or a bond.

Unless stated otherwise, in the present disclosure of the epothilones of formula (I) “lower” means not more than 7 carbon atoms, preferably not more than 4 carbon atoms.

A compound of formula (I),

wherein
A represents O;
R is hydrogen; and
Z is O,
   is known as epothilone A.

A compound of formula (I),

wherein
A represents O;
R is methyl; and
Z is O,
   is known as epothilone B.

A compound of formula (I),

wherein
A represents O;
R is hydrogen; and
Z is a bond,
   is known as epothilone C.

A compound of formula (I),

wherein
A represents O;
R is methyl; and
Z is a bond,
   is known as epothilone D.

Epothilone derivatives of formula (I),

wherein
A represents O or NR_{N}, wherein R_{N} is hydrogen or lower alkyl;
R is hydrogen or lower alkyl; and
Z is O or a bond,
   and methods for the preparation of such epothilone derivatives are, in particular, generically and specifically disclosed in the patents and patent applications WO 93/10121, U.S. Pat. No. 6,194,181, WO 98/25929, WO 98/08849, WO 99/43653, WO 98/22461 and WO 00/31247 in each case, in particular, in the compound claims and the final products of the working examples, the subject matter of the final products, the pharmaceutical preparations and the claims is hereby incorporated into the present application by reference to this publications. Comprised are likewise the corresponding stereoisomers, as well as the corresponding crystal modifications, e.g., solvates and polymorphs, which are disclosed therein. Epothilone derivatives of formula (I′) or (I), especially epothilone B, can be administered as part of pharmaceutical compositions which are disclosed in WO 99/39694.

The protein tyrosine kinase inhibitors of the present invention are described in WO 03/013541, which is incorporated by reference, and are 7H-pyrrolo[2,3-d]pyrimidine derivatives of formula (II)

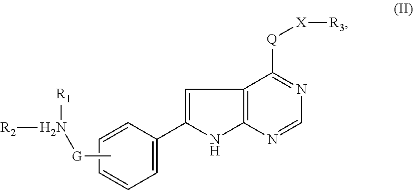

wherein
R₁ and R₂ are, each independently of the other hydrogen, unsubstituted or substituted alkyl or cycloalkyl, a heterocyclic radical bonded via a ring carbon atom or a radical of the formula R₄—Y—(C═Z)—,
wherein
   R₄ is unsubstituted, mono- or di-substituted amino or a heterocyclic radical;
   Y is either not present or lower alkyl; and
   Z is oxygen, sulfur or imino, with the proviso that R₁ and R₂ are not both hydrogen, or
R₁ and R₂, together with the nitrogen atom to which they are attached, form a heterocyclic radical;
R₃ is a heterocyclic radical or an unsubstituted or substituted aromatic radical;
G is C₁-C₇alkylene, —C(═O)— or C₁-C₆alkylene-C(═O)—, wherein the carbonyl group is attached to the NR₁R₂ moiety;
Q is —NH— or —O—, with the proviso that Q is —O— if G is —C(═O)— or C₁-C₆alkylene-C(═O)—; and
X is either not present or C₁-C₇alkylene, with the proviso that a heterocyclic radical R₃ is bonded via a ring carbon atom if X is not present;
   or a salt of the compounds.

The general terms, used hereinbefore and hereinafter in regard to the protein tyrosine kinase inhibitors of formula (II), preferably have within the context of this disclosure the following meanings, unless otherwise indicated.

Where the plural form is used for compounds, salts and the like, this is taken to mean also a single compound, salt or the like.

Where compounds of formula (II) are mentioned which can form tautomers, it is meant to include also the tautomers of such compounds of formula (II). In particular, tautomerism occurs, e.g., for compounds of formula (II) which contain a 2-hydroxy-pyridyl radical. In such compounds the 2-hydroxy-pyridyl radical can also be present as pyrid-2(1H)-on-yl.

Asymmetric carbon atoms of a compound of formula (II) that are optionally present may exist in the (R), (S) or (R,S) configuration, preferably in the (R) or (S) configuration. Substituents at a double bond or a ring may be present in cis-(═Z-) or trans (=E-) form. The compounds may thus be present as mixtures of isomers or preferably as pure isomers.

Preferably alkyl of formula (II) contains up to 20 carbon atoms and is most preferably lower alkyl.

The prefix “lower” of formula (II( denotes a radical having up to and including a maximum of 7 carbon atoms, especially up to and including a maximum of 4 carbon atoms, the radicals in question being either unbranched or branched with single or multiple branching. Lower alkyl is, e.g., methyl, ethyl, n-propyl, isopropyl, n-butyl, isobutyl, sec-butyl, tert-butyl, n-pentyl, isopentyl, neopentyl, n-hexyl or n-heptyl.

Alkyl R₁ and R₂ of formula (II), independently of each other, are preferably methyl, ethyl, isopropyl or tert-butyl, especially methyl or ethyl.

Lower alkyl Y of formula (II) is preferably methyl, ethyl or propyl.

Lower alkoxy of formula (II) is, e.g., ethoxy or methoxy, especially methoxy.

Substituted alkyl of formula (II) is preferably lower alkyl, as defined above, where one or more substituents, preferably one substituent may be present, such as, e.g., amino, N-lower alkylamino, N,N-di-lower alkylamino, N-lower alkanoylamino, N,N-di-lower alkanoylamino, hydroxy, lower alkoxy, lower alkanoyl, lower alkanoyloxy, cyano, nitro, carboxy, lower alkoxycarbonyl, carbamoyl, N-lower alkyl-carbamoyl, N,N-di-lower alkyl-carbamoyl, amidino, guanidino, ureido, mercapto, lower alkylthio, halogen or a heterocyclic radical.

Substituted alkyl R₁ and R₂ of formula (II) are, independently of each other, preferably hydroxy-lower alkyl, N,N-di-lower alkylamino-lower alkyl or morpholinyl-lower alkyl.

Preferably unsubstituted or substituted cycloalkyl R₁ or R₂ of formula (II) contains from 3 carbon atoms, up to 20 carbon atoms, and is especially unsubstituted or also substituted C₃-C₆cycloalkyl, wherein the substituents are selected from, e.g., unsubstituted or substituted lower alkyl, amino, N-lower alkylamino, N,N-di-lower alkylamino, N-lower alkanoylamino, N,N-di-lower alkanoylamino, hydroxy, lower alkoxy, lower alkanoyl, lower alkanoyloxy, cyano, nitro, carboxy, lower alkoxycarbonyl, carbamoyl, N-lower alkyl-carbamoyl, N,N-di-lower alkyl-carbamoyl, amidino, guanidino, ureido, mercapto, lower alkylthio, halogen or a heterocyclic radical.

Mono- or di-substituted amino of formula (II) is amino substituted by one or two radicals selected independently of one another from, e.g., unsubstituted or substituted lower alkyl.

Di-substituted amino R₄ of formula (II) is preferably N,N-di-lower alkylamino, especially N,N-dimethylamino or N,N-diethylamino.

A heterocyclic radical of formula (II) contains especially up to 20 carbon atoms and is preferably a saturated or unsaturated monocyclic radical having from 4 or 8 ring members and from 1-3 heteroatoms, which are preferably selected from nitrogen, oxygen and sulfur, or a bi- or tri-cyclic radical wherein, e.g., one or two carbocyclic radicals, such as, e.g., benzene radicals, are annellated (fused) to the mentioned monocyclic radical. If a heterocyclic radical contains a fused carbocyclic radical then the heterocyclic radical may also be attached to the rest of the molecule of formula (I) via a ring atom of the fused carbocyclic radical. The heterocyclic radical, including the fused carbocyclic radical(s) if present, is optionally substituted by one or more radicals, preferably by one or two radicals, such as, e.g., unsubstituted or substituted lower alkyl, amino, N-lower alkylamino, N,N-di-lower alkylamino, N-lower alkanoylamino, N,N-di-lower alkanoylamino, hydroxy, lower alkoxy, lower alkanoyl, lower alkanoyloxy, cyano, nitro, carboxy, lower alkoxycarbonyl, carbamoyl, N-lower alkyl-carbamoyl, N,N-di-lower alkyl-carbamoyl, amidino, guanidino, ureido, mercapto, lower alkylthio or halogen.

Most preferably, a heterocyclic radical of formula (II) is pyrrolidinyl, piperidyl, lower alkyl-piperazinyl, di-lower alkyl-piperazinyl, morpholinyl, tetrahydropyranyl, pyridyl, pyridyl substituted by hydroxy or lower alkoxy or benzodioxolyl, especially pyrrolidinyl, piperidyl, lower alkyl-piperazinyl, di-lower alkyl-piperazinyl or morpholinyl.

A heterocyclic radical R₁ or R₂ of formula (II) is as defined above for a heterocyclic radical with the proviso that it is bonded to the rest of the molecule of formula (II) via a ring carbon atom. Preferably, a heterocyclic radical R₁ or R₂ is lower alkyl-piperazinyl or especially preferred tetrahydropyranyl. If one of the two radicals R₁ and R₂ represents a heterocyclic radical, the other is preferably hydrogen.

A heterocyclic radical R₃ of formula (II) is as defined above for a heterocyclic radical with the proviso that it is bonded to Q via a ring carbon atom if X is not present. Preferably, a heterocyclic radical R₃ is benzodioxolyl, pyridyl substituted by hydroxy or lower alkoxy, or especially preferred indolyl substituted by halogen and lower alkyl. If R₃ is pyridyl substituted by hydroxy then the hydroxy group is preferably attached to a ring carbon atom adjacent to the ring nitrogen atom.

A heterocyclic radical R₄ of formula (II) is as defined above for a heterocyclic radical and is preferably pyrrolidinyl, piperidyl, lower alkyl-piperazinyl, morpholinyl or pyridyl.

If R₁ and R₂ of formula (II), together with the nitrogen atom to which they are attached, form a heterocyclic radical, the heterocyclic radical is as defined above for a heterocyclic radical and represents preferably pyrrolidinyl, piperidyl, lower alkyl-piperazinyl, di-lower alkyl-piperazinyl or morpholinyl.

An unsubstituted or substituted aromatic radical R₃ of formula (II) has up to 20 carbon atoms and is unsubstituted or substituted, e.g., in each case unsubstituted or substituted phenyl. Preferably, an unsubstituted aromatic radical R₃ is phenyl. A substituted aromatic radical R₃ is preferably phenyl substituted by one or more substituents selected independently of one another from the group consisting of unsubstituted or substituted lower alkyl, amino, N-lower alkylamino, N,N-di-lower alkylamino, N-lower alkanoylamino, N,N-di-lower alkanoylamino, hydroxy, lower alkoxy, lower alkanoyl, lower alkanoyloxy, cyano, nitro, carboxy, lower alkoxycarbonyl, carbamoyl, N-lower alkyl-carbamoyl, N,N-di-lower alkyl-carbamoyl, amidino, guanidino, ureido, mercapto, lower alkylthio and halogen. Most preferably, a substituted aromatic radical R₃ is phenyl substituted by one or more radicals selected independently of one another from the group consisting of lower alkyl, amino, hydroxy, lower alkoxy, halogen and benzyloxy.

Halogen of formula (II) is primarily fluoro, chloro, bromo or iodo, especially fluoro, chloro or bromo.

C₁-C₇Alkylene of formula (II) may be branched or unbranched and is, in particular, C₁-C₃alkylene.

C₁-C₇Alkylene G of formula (II) is preferably C₁-C₃alkylene, most preferably methylene (—CH₂—).

If G of formula (II) is not C₁-C₇alkylene, it preferably represents —C(═O)—.

C₁-C₇Alkylene X of formula (II) is preferably C₁-C₃alkylene, most preferably methylene (—CH₂—) or ethan-1,1-diyl (—CH(CH₃)—).

Q of formula (II) is preferably —NH—.

Z of formula (II) is preferably oxygen or sulfur, most preferably oxygen.

In one embodiment, a particularly preferred protein tyrosine kinase inhibitor for use in the invention is {6-[4-(4-ethyl-piperazin-1-ylmethyl)-phenyl]-7H-pyrrolo[2,3-c]pyrimidin-4-yl}-(1-[phenyl-ethyl)-amine or a pharmaceutically acceptable salt thereof.

Rapamycin is a known macrolide antibiotic produced by Streptomyces hygroscopicus. Suitable derivatives of rapamycin of the present invention include, e.g., compounds of formula (III)

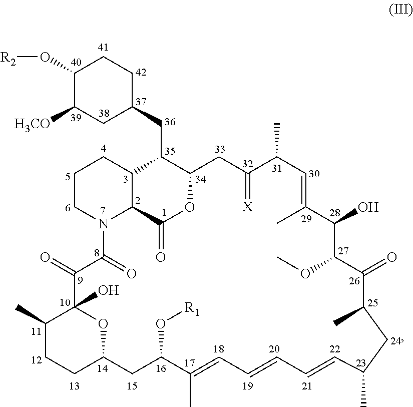

wherein
R₁ is CH₃ or C₃-C₆alkynyl;
R₂ is H or —CH₂—CH₂—OH; and
X is ═O, (H,H) or (H,OH),
   provided that R₂ is other than H when X is ═O and R₁ is CH₃.

Compounds of formula (III) are disclosed, e.g., in WO 94/09010, WO 95/16691 or WO 96/41807, which are incorporated herein by reference. They may be prepared as disclosed or by analogy to the procedures described in these references.

In one embodiment, a particularly preferred derivative of rapamycin of formula (III) is one, where
R₁ is CH₃,
R₂ is —CH₂—CH₂—OH; and
X is O.

Furthermore, the structure of the active agents mentioned herein by name may be taken from the actual edition of the standard compendium “The Merck Index” or from databases, e.g., Patents International, e.g., IMS World Publications. The corresponding content thereof is hereby incorporated by reference. Any person skilled in the art is fully enabled, based on these references, to manufacture and test the pharmaceutical indications and properties in standard test models, both in vitro and in vivo.

In view of the close relationship between the novel compounds in free form and in the form of their salts, including those salts that can be used as intermediates, e.g., in the purification or identification of the novel compounds, hereinbefore and hereinafter any reference to the free compounds is to be understood as referring also to the corresponding salts, as appropriate and expedient.

The compounds used as combination partners (a) and (b), and optionally (c), disclosed herein can be prepared and administered as described in the cited documents, respectively.

It will be understood that references to the combination partners (a) and (b), and optionally (c), are meant to also include the pharmaceutically acceptable salts. If these combination partners (a) and (b), and optionally (c), have, e.g., at least one basic center, they can form acid addition salts. Corresponding acid addition salts can also be formed having, if desired, an additionally present basic center. The combination partners (a) and (b), and optionally (c), having an acid group, e.g., COOH, can also form salts with bases. The combination partner (a) and (b), and optionally (c), or a pharmaceutically acceptable salt thereof may also be used in form of a hydrate or include other solvents used for crystallization.

A combination which comprises:

(a) an epothilone derivative of formula (I),

in which compound
A represents O or NR_{N}, wherein R_{N} is hydrogen or lower alkyl;
R is hydrogen or lower alkyl; and
Z is O or a bond; and

(b) a protein tyrosine kinase inhibitor of formula (II), preferably {6-[4-(4-ethyl-piperazin-1-ylmethyl)-phenyl]-7H-pyrrolo[2,3-d]pyrimidin-4-yl}-(1-[phenyl-ethyl)-amine and optionally (a) a derivative of rapamycin as defined in formula (III), preferably

where
R₁ is CH₃;
R₂ is —CH₂—CH₂—OH; and
X is O,
   in which the active ingredients are present in each case in free form or in the form of a pharmaceutically acceptable salt and optionally at least one pharmaceutically acceptable carrier, will be referred to hereinafter as a COMBINATION OF THE INVENTION.

The COMBINATIONS OF THE INVENTION inhibits the growth of solid tumors, but also liquid tumors. The nature of proliferative diseases like solid tumor diseases is multifactorial. Under certain circumstances, drugs with different mechanisms of action may be combined. However, just considering any combination of drugs having different mode of action does not necessarily lead to combinations with advantageous effects.

It is one objective of this invention to provide a pharmaceutical composition comprising a quantity, which is jointly therapeutically effective against a proliferative disease comprising the COMBINATION OF THE INVENTION. In this composition, the combination partners (a) and (b), and optionally (c), can be administered together, one after the other or separately in one combined unit dosage form or in two separate unit dosage forms. The unit dosage form may also be a fixed combination.

The pharmaceutical compositions according to the invention can be prepared in a manner known per se and are those suitable for enteral, such as oral or rectal; and parenteral administration to mammals (warm-blooded animals), including man, comprising a therapeutically effective amount of at least one pharmacologically active combination partner alone or in combination with one or more pharmaceutically acceptable carries, especially suitable for enteral or parenteral application.

The novel pharmaceutical composition contain, e.g., from about 10% to about 100%, preferably from about 20% to about 60%, of the active ingredients. Pharmaceutical preparations for the combination therapy for enteral or parenteral administration are, e.g., those in unit dosage forms, such as sugar-coated tablets, tablets, capsules or suppositories, and furthermore ampoules. If not indicated otherwise, these are prepared in a manner known per se, e.g., by means of conventional mixing, granulating, sugar-coating, dissolving or lyophilizing processes. It will be appreciated that the unit content of a combination partner contained in an individual dose of each dosage form need not in itself constitute an effective amount since the necessary effective amount can be reached by administration of a plurality of dosage units.

In preparing the compositions for oral dosage form, any of the usual pharmaceutical media may be employed, such as, e.g., water, glycols, oils, alcohols, flavoring agents, preservatives, coloring agents; or carriers, such as starches, sugars, microcrystalline cellulose, diluents, granulating agents, lubricants, binders, disintegrating agents and the like in the case of oral solid preparations, such as, e.g., powders, capsules and tablets, with the solid oral preparations being preferred over the liquid preparations. Because of their ease of administration, tablets and capsules represent the most advantageous oral dosage unit form in which case solid pharmaceutical carriers are obviously employed.

In particular, a therapeutically effective amount of each of the combination partners of the COMBINATION OF THE INVENTION may be administered simultaneously or sequentially and in any order, and the components may be administered separately or as a fixed combination. For example, the method of delay of progression or treatment of a proliferative disease according to the invention may comprise:
(a) administration of the first combination partner in free or pharmaceutically acceptable salt form; and
(b) administration of the second combination partner in free or pharmaceutically acceptable salt form; and optionally
(c) administration of the third combination partner in free or pharmaceutically acceptable salt form, simultaneously or sequentially in any order, in jointly therapeutically effective amounts, preferably in synergistically effective amounts, e.g., in daily dosages corresponding to the amounts described herein.

The individual combination partners of the COMBINATION OF THE INVENTION can be administered separately at different times during the course of therapy or concurrently in divided or single combination forms. Furthermore, the term administering also encompasses the use of a pro-drug of a combination partner that convert in vivo to the combination partner as such. The instant invention is therefore to be understood as embracing all such regimes of simultaneous or alternating treatment and the term “administering” is to be interpreted accordingly.

The effective dosage of each of the combination partners employed in the COMBINATION OF THE INVENTION may vary depending on the particular compound or pharmaceutical composition employed, the mode of administration, the condition being treated and the severity of the condition being treated. Thus, the dosage regimen the COMBINATION OF THE INVENTION is selected in accordance with a variety of factors including the route of administration and the renal and hepatic function of the patient. A physician, clinician or veterinarian of ordinary skill can readily determine and prescribe the effective amount of the single active ingredients required to prevent, counter or arrest the progress of the condition. Optimal precision in achieving concentration of the active ingredients within the range that yields efficacy without toxicity requires a regimen based on the kinetics of the active ingredients' availability to target sites. This involves a consideration of the distribution, equilibrium, and elimination of the active ingredients.

If the warm-blooded animal is a human, the dosage of a compound of formula (I′) or (I) is preferably in the range of about 0.25-75 mg/m², preferably 0.5-50 mg/m², e.g., 2.5, mg/m² once weekly for 2-4 weeks, e.g., 3 weeks, followed by 6-8 days off in the case of an adult patient.

Epothilone B is preferably administered in a dose which is calculated according to the formula (IV)

single dose(mg/m²)=(0.1 to y)×N (IV)

wherein
N is the number of weeks between treatments; and
y is 6,
   wherein epothilone B is administered in more than one treatment cycle after an interval of 1-6 weeks after the preceding treatment.

In one preferred embodiment of the invention, epothilone B is administered weekly in a dose that is between about 0.1-6 mg/m², preferably between 0.1 and 3 mg/m², e.g., 2.5 or 3.0 mg/m², for 3 weeks after an interval of 1-6 weeks, especially an interval of 1 week, after the preceding treatment. In another embodiment of the invention said epothilone B is preferably administered to a human every 18-24 days in a dose that is between about 0.3 and 12 mg/m².

The protein tyrosine kinase inhibitors of formula (II) of the present invention can be administered for an individual having a bodyweight of about 70 kg the daily dose from approximately 0.1 g to approximately 5 g, preferably from approximately 0.5 g to approximately 2 g, of a compound of the present invention.

In general, results are achieved on administration of a derivative rapamycin of formula (II) of the present invention at daily dosage rates of the order of about 0.1-25 mg as a single dose or in divided doses. Suitable unit dosage forms for derivatives of rapamycin of the present invention for oral administration comprise from ca. 0.05-10 mg active ingredient.

In a preferred embodiment of the invention, the COMBINATION OF THE INVENTION comprises a protein tyrosine kinase inhibitor which is {6-[4-(4-ethyl-piperazin-1-ylmethyl)-phenyl]-7H-pyrrolo[2,3-c]pyrimidin-4-yl}-(1-[phenyl-ethyl)-amine or a pharmaceutically acceptable salt thereof.

In another embodiment of the invention, the COMBINATION OF THE INVENTION comprises a derivative of rapamycin of formula (III),

wherein
R₁ is CH₃;
R₂ is —CH₂—CH₂—OH; and
X is O.

The COMBINATION OF THE INVENTION can be a combined preparation or a pharmaceutical composition.

Moreover, the present invention relates to a method of treating a warm-blooded animal having a proliferative disease, in particular, cancer, comprising administering to the animal a COMBINATION OF THE INVENTION in a quantity which is jointly therapeutically effective against a proliferative disease and in which the combination partners can also be present in the form of their pharmaceutically acceptable salts.

Furthermore, the present invention pertains to the use of a COMBINATION OF THE INVENTION for the delay of progression or treatment of a proliferative disease and for the preparation of a medicament for the delay of progression or treatment of a proliferative disease.

Additionally, the present invention pertains to the use of epothilone of formula (I′) or (I) in combination with a protein tyrosine kinase inhibitor of formula (II) and optionally a derivative of rapamycin of formula (III) for the preparation of a medicament for the delay of progression or treatment of a proliferative disease.

Moreover, the present invention provides a commercial package comprising as active ingredients COMBINATION OF THE INVENTION, together with instructions for simultaneous, separate or sequential use thereof in the delay of progression or treatment of a proliferative disease.

The following Examples illustrate the invention described above; they are not, however, intended to limit the scope of the invention in any way. The beneficial effects of the COMBINATION OF THE INVENTION can also be determined by other test models known as such to the person skilled in the pertinent art.

1. A combination which comprises:
(a) an epothilone derivative of formula (I′) 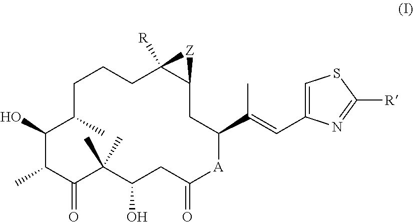
wherein A represents O or NR_{N}, wherein R_{N} is hydrogen or lower alkyl, R is hydrogen or lower alkyl, R′ is methyl, methoxy, ethoxy, amino, methylamino, dimethylamino, aminomethyl or methylthio, and Z is O or a bond, in free form or in the form of a pharmaceutically acceptable salt; and
(b) a protein tyrosine kinase inhibitor, in which the active ingredients (a) and (b) are present in each case in free form or in the form of a pharmaceutically acceptable salt and optionally at least one pharmaceutically acceptable carrier; for simultaneous, separate or sequential use.

2. The combination as claimed in claim 1, wherein the protein tyrosine kinase inhibitor is a compound of the following formula (II) 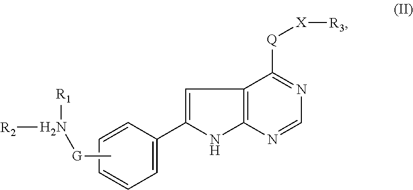
wherein
R₁ and R₂ are, each independently of the other hydrogen, unsubstituted or substituted alkyl or cycloalkyl, a heterocyclic radical bonded via a ring carbon atom, or a radical of the formula R₄—Y—(C═Z)—,
wherein
R₄ is unsubstituted, mono- or di-substituted amino or a heterocyclic radical;
Y is either not present or lower alkyl; and
Z is oxygen, sulfur or imino,
with the proviso that R₁ and R₂ are not both hydrogen, or
R₁ and R₂, together with the nitrogen atom to which they are attached form, a heterocyclic radical;
R₃ is a heterocyclic radical or an unsubstituted or substituted aromatic radical;
G is C₁-C₇alkylene, —C(═O)— or C₁-C₆alkylene-C(═O)—, wherein the carbonyl group is attached to the NR₁R₂ moiety;
Q is —NH— or —O—, with the proviso that Q is —O— if G is —C(═O)— or C₁-C₆alkylene-C(═O)—; and
X is either not present or C₁-C₇alkylene, with the proviso that a heterocyclic radical R₃ is bonded via a ring carbon atom if X is not present;
or a salt of the compounds.

3. The combination as claimed in claim 2, wherein the protein tyrosine kinase inhibitor is {6-[4-(4-ethyl-piperazin-1-ylmethyl)-phenyl]-7H-pyrrolo[2,3-d]pyrimidin-4-yl}-(1-[phenyl-ethyl)-amine or a pharmaceutically acceptable salt thereof.

4. The combination as claimed in claim 1, further comprising a rapamycin derivative in free form or in the form of a pharmaceutically acceptable salt.

5. The combination as claimed in claim 4 wherein the rapamycin derivative is a compound of formula (III) 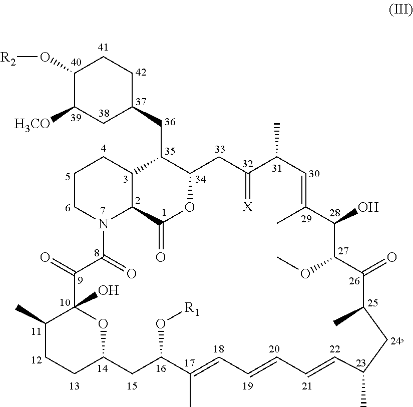
wherein
R₁ is CH₃ or C₃-C₆alkynyl;
R₂ is H or —CH₂—CH₂—OH; and
X is ═O, (H,H) or (H,OH), provided that R₂ is other than H when X is ═O and R₁ is CH₃;
and a pharmaceutically acceptable salt thereof.

6. The combination as claimed in claim 5,
wherein
R₁ is CH₃;
R₂ is —CH₂—CH₂—OH; and
X is O.

7. The combination as claimed in claim 1, comprising an epothilone derivative of formula (I), 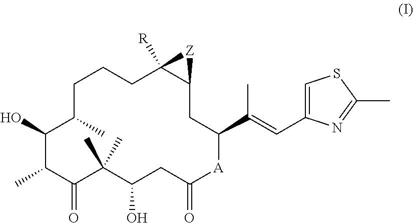
wherein
A represents O;
R is lower alkyl or hydrogen; and
Z is O or a bond.

8. The combination as claimed in claim 1, which is a combined preparation or a pharmaceutical composition.

9. Method of treating a warm-blooded animal having a proliferative disease comprising administering to the animal a combination according to claim 1 in a quantity which is jointly therapeutically effective against a proliferative disease and in which the compounds can also be present in the form of their pharmaceutically acceptable salts.

10. The method of treating as claimed in claim 9, wherein the proliferative disease is cancer.

11. The method of treating as claimed in claim 9, wherein the cancer is breast cancer, lung cancer, glioma, prostate cancer, ovarian cancer, colorectal cancer, pancreatic cancer, hepatic cancer and renal cancer.

12. A pharmaceutical composition comprising a quantity which is jointly therapeutically effective against a proliferative disease of a pharmaceutical combination as claimed in claim 1 and at least one pharmaceutically acceptable carrier.

13. The combination as claimed in claim 1, for use in the delay of progression or treatment of a proliferative disease.

14. Use of a combination as claimed in claim 1, for the preparation of a medicament for the treatment of a proliferative disease.

15. A commercial package comprising:
(a) an epothilone derivative of formula (I) 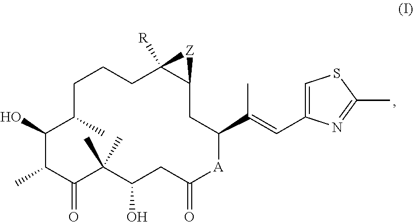
wherein
A represents O or NR_{N}, wherein R_{N} is hydrogen or lower alkyl;
R is hydrogen or lower alkyl; and
Z is O or a bond; and
(b) a protein tyrosine kinase inhibitor; together with instructions for simultaneous, separate or sequential use thereof in the delay of progression or treatment of a proliferative disease.

16. The commercial package as claimed in claim 15, wherein the protein tyrosine kinase inhibitor is a compound of the following formula (II) 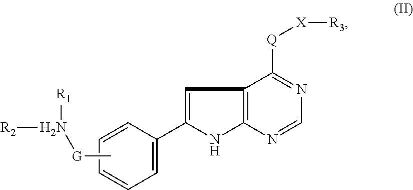
wherein
R₁ and R₂ are, each independently of the other hydrogen, unsubstituted or substituted alkyl or cycloalkyl, a heterocyclic radical bonded via a ring carbon atom, or a radical of the formula R₄—Y—(C═Z)—,
wherein
R₄ is unsubstituted, mono- or di-substituted amino or a heterocyclic radical;
Y is either not present or lower alkyl; and
Z is oxygen, sulfur or imino, with the proviso that R₁ and R₂ are not both hydrogen, or
R₁ and R₂, together with the nitrogen atom to which they are attached, form a heterocyclic radical;
R₃ is a heterocyclic radical or an unsubstituted or substituted aromatic radical;
G is C₁-C₇alkylene, —C(═O)— or C₁-C₆alkylene-C(═O)—, wherein the carbonyl group is attached to the NR₁R₂ moiety;
Q is —NH— or —O—, with the proviso that Q is —O— if G is —C(O)— or C₁-C₆alkylene-C(═O)—; and
X is either not present or C₁-C₇alkylene, with the proviso that a heterocyclic radical R₃ is bonded via a ring carbon atom if X is not present;
or a salt of the compounds.

17. The commercial package as claimed in claim 16, wherein the protein tyrosine kinase inhibitor is {6-[4-(4-ethyl-piperazin-1-ylmethyl)-phenyl]-7H-pyrrolo[2,3-c]pyrimidin-4-yl}-(1-[phenyl-ethyl)-amine or a pharmaceutically acceptable salt thereof.

18. The commercial package as claimed in claim 15, further comprising a rapamycin derivative.

19. The commercial package as claimed in claim 18, wherein the rapamycin derivative is a compound of formula (III) 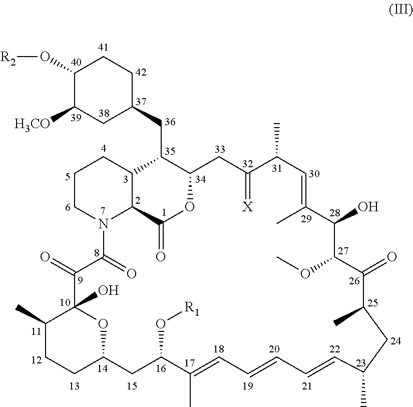
wherein
R₁ is CH₃ or C₃-C₆alkynyl;
R₂ is H or —CH₂—CH₂—OH; and
X is ═O, (H,H) or (H,OH), provided that R₂ is other than H when X is ═O and R₁ is CH₃;
and a pharmaceutically acceptable salt thereof.

20. The commercial package as claimed in claim 19,
wherein
R₁ is CH₃;
R₂ is —CH₂—CH₂—OH; and
X is O.
